SEQUENCE LISTING (14 sequences)

SEQ ID NO: 1 - protein, 245 aa, Semibalanus balanoides
  MISC_FEATURE (146)..(146): X is A or V
MKLLLFVCVCALAAAEQQVVVPTTGQLAVGLQHLYRRVMHKVAPKELEITDALHIQWRNGMLSGPPFRSPVKGTVDGVEYPTFSYINSLINPGQKGVFGMWTKHDTSDLVLTYHYGGSMKVHMLTDGGIHKEAILGNPTDRKHKEXKFVVMIPKNTYCIFESLTEDESTFKSYIAIPAWNKENDHRFTQEQMTAKFPDFTELFKELAKPGVHDDHDDHHDDHHDDHHDERRRYGGNHGRFAGRKH

SEQ ID NO: 2 - DNA, 740 bp, Semibalanus balanoides
atgaagctgctactgtttgtctgcgtgtgtgcgctggctgcggccgaacagcaggtggttgtaccgacgactggccagctggccgtcggtctgcaacacctgtaccgtcgctgttatgcacgaggtggccccgaaagagctggagatcaccgacgccctccacatccagtggcgcaacggcatgctgtccggcccgcccttcaggtctccggtgaagggaactgttgacggagttgagtacccgaccttctcgtacatcaacagccttatcaaccctggtcagaagggtgtctttggaatgtggaccaaacacgatacctctgatctggtgctcacctaccactacggtggctccatgaaggttcatatgctgactgacggcggaattcacaaggaggccatccttggcaaccctacggaccgcaagcataaggaggctcaaattcgtcgtcatgatccctaagaacacctactgcatcttcgagtctctcacggaggatgagtcgaccttcaaatcctacatcgcaatccccgcctggaacaaggagaacgaccaccgcttcacccaggagcagatgacggccaagttcccagacttcaccgagttgttcaaggagcttgccaagccgggcgttcacgatgatcatgatgaccatcatgatgaccatcatgatgaccatcatgatgagcgtcgtcgttacggcggcaaccatggacgttttgctggcaggaagcactaa

SEQ ID NO: 3 - DNA, 30 bp, Semibalanus balanoides
cagcaggtsgtsgtsccsacsacsggccag

SEQ ID NO: 4 - DNA, 24 bp, Semibalanus balanoides
ggtcagatcttggatcctgtcgac

SEQ ID NO: 5 - DNA, 25 bp, Semibalanus balanoides
tccaaagacacccttctgaccaggg

SEQ ID NO: 6 - DNA, 25 bp, Semibalanus balanoides
gctgttgatgtacgagaaggtcggg

SEQ ID NO: 7 - DNA, 30 bp, Semibalanus balanoides
atgaagctgctactgtttgtctgcgtgtgt

SEQ ID NO: 8 - DNA, 27 bp, Semibalanus balanoides
gtgcttcctgccagcaaaacgtccatg

SEQ ID NO: 9 - protein, 56 aa, Semibalanus balanoides
  SIGNAL (1)..(15)
MRVILFAMLIGGSLACQNRLETLVQEATGNAGDLSTNVHEECNSQVGTFNAVHAPQ

SEQ ID NO: 10 - DNA, 171 bp, Semibalanus balanoides
atgcgcgtcattctgttcgcgatgctgattggcggctctctggcctgccagaaccggctggagacgctcgtccaggaagccaccgggaatgccggagacctttccacaaatgtgcacgaggaatgcaactcccaggtgggcacgttcaatgcggtgcacgcgccccagtga

SEQ ID NO: 11 - DNA, 32 bp, Semibalanus balanoides
tggcagaaccgsctsgaracsctsgtscagga

SEQ ID NO: 12 - DNA, 24 bp, Semibalanus balanoides
ggtcagatcttggatcctgtcgac

SEQ ID NO: 13 - DNA, 24 bp, Semibalanus balanoides
ggagttgcattcctcgtgcacatt

SEQ ID NO: 14 - DNA, 17 bp, Semibalanus balanoides
gcattcccggtggctta

(19) United States
(12) Patent Application Publication
(10) Pub. No.: US 2003/0143673 A1
(43) Pub. Date: Jul. 31, 2003

(54) Barnacle adhesion proteins

(76) Inventors: David L. Kaplan, Concord, MA (US); Paul Gatenholm, Kullavik (SE); Karl Mattias Berglin, De Geerg (SE); Joseph David Platko, Merrimack, NH (US); Lauren Rebecca Pepper, Westfield, NJ (US); Alyssa Vanita Ngangan, Nahant, MA (US)

Correspondence Address: BLANK ROME COMISKY & MCCAULEY, LLP, 900 17TH STREET, N.W., SUITE 1000, WASHINGTON, DC, 20006, US

(21) Appl. No.: 10/134,507

(22) Filed: Apr. 30, 2002

Related U.S. Application Data
(63) Continuation-in-part of application No. 10/055,904, filed on Jan. 28, 2002.

(51) Int. Cl. C12P 21/02; C12N 5/06; C07K 14/435; C07H 21/04; C07K 16/44

(52) U.S. Cl. 435/69.1; 435/320.1; 435/325; 530/395; 536/23.5; 530/388.1

(57) ABSTRACT

The proteins involved in barnacle are useful in devising high-strength protein-based adhesives capable of curing under water, coatings for prosthetic implants to serve as an interface between the prosthetic and the bone or other tissue, and methods of preventing biofouling of underwater surfaces. DNA and amino acid sequences of the adhesion proteins are provided and isolated nucleic acid sequences and isolated proteins, vectors comprising such isolated nucleic acid sequences as well as microorganisms comprising such vectors and capable of expressing a barnacle adhesive protein are also provided.

### FIELD OF THE INVENTION

The present invention relates to proteins involved in barnacle adhesion which, according to the invention, are used to devise protein-based adhesives capable of curing under water or in the body, coatings for prosthetic implants to serve as an interface between the prosthetic and the bone or other tissues, and methods of preventing biofouling of underwater surfaces.

### BACKGROUND OF THE INVENTION

Various marine organisms are able to attach themselves to underwater surfaces, which is a part of their normal life cycle. In particular, barnacles and mussels have attracted much attention for their fouling properties and their tenacious underwater adhesion by a proteinaceous secretion. Various chemical and mechanical treatments have been used to remove barnacles and mussels from ships and piers and/or prevent them from attaching to them. Many current methods for controlling fouling, and in turn invertebrate adhesion, require the use of toxic components such as copper and tin containing compounds. In many cases mechanical cleaning does not effectively remove the barnacles, and even in cases where it does, rapid re-colonization is often a problem.

The mechanisms by which barnacles and muscles attach themselves to surfaces are not well understood; however, it is known that these organisms produce an adhesive to facilitate attachment to rocks and other surfaces. The adhesives produced by mussels and barnacles represent some of the strongest natural adhesive known. They appear to be protein based; and the system used by the blue mussel, Mytilus edulis, has been studied. The proteins in this system comprise hexa- and decapeptide repeats and a variety of post translational modifications, involved in the tanning (curing) of the protein, are putatively responsible for the noncovalent adhesion to the surface substrate (see Taylor et al., 1997, In Protein-Based Materials, eds McGrath et al., Birkhauser, Boston, Mass., pp. 17-250, for a review).

Unlike the mussel adhesion system, the adhesion systems of barnacles have been the subject of relatively few studies. From the data available to date, it appears that the mechanisms involved in barnacle cement adhesion and curing are significantly different than that of the mussel. Walker (1970), Marine Biology, 7:239-248, first demonstrated that adult barnacle cement contains proteins using the histochemistry of three barnacles, Eliminius modestus, Balanus balanoides, and Balanus hameri. Following this initial observation, Walker, 1972, Journal of the Marine Biological Association UK, 52:429-435, reported the biochemical contents of the cements of B. hameri and B. crentaus. The cements of these two species were found to have similar amino acid compositions, including a high level of cysteine. The data suggested a role for disulfide bond formation in barnacle cement, which was confirmed by Naldrett, 1993, Journal of the Marine Biological Association UK, 73:689-702. Naldrett reported on the solubilization of adhesion plaques from B. perforatus, B. crenatus, and B. balanoides using sodium dodecyl sulfate (SDS) and 2-mercaptoethanol. Others also confirmed the observation that disulfide bonds are involved in barnacle cement formation through solubilization of barnacle cement using chemical denaturants that reduced disulfide bonds (Barnes et al., 1976, Journal of Experimental Marine Biology and Ecology, 25:263-271; Yan et al., 1981, Oceanologia et Limmologia Sinica, 12:125-132; and Kamino et al., 2000, Journal of Biological Chemistry, 275:27360-27365).

The protein components of barnacle cement have also been analyzed through SDS-PAGE. Naldrett reported proteins of molecular weights ≦15 kD, which led to the hypothesis that the lower molecular weight proteins were subunits of the cement and aggregated to form the higher molecular weight proteins.

Naldrett et al, 1997, Marine Biology, 127:629-635, reported five major proteins, from the cement of B. eburneus, identified by SDS-PAGE based on solubilization in SDS and 2-mercaptoethanol at 100° C. These proteins have molecular weights of 7, 22, 36, 52, and 58 kD.

Kamino et al, 1996, Biological Bulletin, 190:403-409, reported and isolated three proteins from the cement of Magabalanus rosa. These proteins have molecular weights of 47, 57, 60 and 180 kD, and the 180 kD protein is believed to be an aggregate of the 57 and 60 kD proteins. All four proteins have the same N-terminal sequence of GKAVTVGTD. The 47 kD protein has also been cloned and sequenced by Kamino et al. (JP1997-47288).

### SUMMARY OF THE INVENTION

The present invention relates to proteins involved in barnacle adhesion. The proteins are secreted for aggregation, polymerization, and/or cross-linking forming the base plate and/or glue attaching the barnacle to underwater surfaces. These proteins are useful in devising protein-based adhesives capable of curing under water, coatings for prosthetic implants to serve as an interface between the prosthetic and the bone or other tissue, and methods of preventing biofouling of underwater surfaces. DNA and amino acid sequences of the adhesion proteins are provided. The invention provides isolated nucleic acid sequences and isolated proteins, vectors comprising such isolated nucleic acid sequences as well as microorganisms comprising such vectors and capable of expressing a barnacle adhesive protein.

### BRIEF DESCRIPTION OF THE FIGURES

FIG. 1 is a SDS-PAGE depicting proteins in the barnacle hemolymph and base plate.

FIG. 2 is a SDS-PAGE showing barnacle proteins after purification with nickel beads.

FIG. 3 is an agarose gel showing a PCR result amplifying the gene for the 35 kD protein.

FIG. 4 show sequence homology of the amino terminus of B. cretanus, B. improvisus and S. Balanoides.

### DETAILED DESCRIPTION OF THE INVENTION

I. Protein Isolation and Characterization

The proteins of the present invention are isolated from the hemolymph and the base plate of the barnacle. The hemolymph is collected from the mantle cavity of the barnacle. Access to the mantle cavity is obtained by prying attached barnacles from the outer shell of mussels with a palette knife. Detached barnacles are inverted and the hemolymph is collected with a pipette device.

To collect protein samples from the base plate, the base plate is solubilized in solution in the presence of reducing agents such as dithiothreitol (DTT), guadinium thiocyanate and/or 2-mercaptoethanol. Most preferably, guadinium thiocyanate is used. The base plate of the barnacle is removed with a razor blade and placed into a solubilization buffer (250 grams of guandinium thiocyanate (Fluka), 293 ml of diethylpyrocarbonate (DEPC) water, 17.6 ml of 0.75 M sodium citrate, 26.4 ml of 10% Sarcosyl (IBI, Inc.) with 7 μl of 2-mercaptoethanol per milliliter of solution (0.09M final)) for 18 hours.

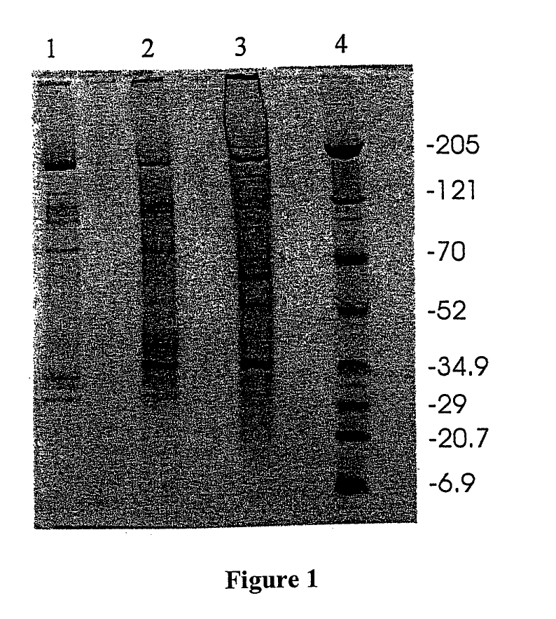

Protein samples from the hemolymph and the base plate are resolved by SDS-PAGE where the proteins are separated based on molecular weight. The procedure for SDS-PAGE is well-known in the art. Preferably, the gel contains about 3 to about 40 percent polyacrylamide, more preferable about 3 to about 20 percent polyacrylamide, and most preferable about 12 to about 17 percent polyacrylamide. The SDS-PAGE can also be a gradient gel. After electrophoresis, the protein bands can be visualized by staining the gel with Coomassie blue or silver stain. FIG. 1 shows a SDS-PAGE of the hemolymph and the base plate proteins.

For sequencing, the proteins on the SDS-PAGE are transferred to a PVDF membrane and visualized by Ponceau staining. The protein bands are then cut from the membranes and sequenced.

The proteins are sequenced according to methods known in the art. The most common method available for protein sequencing is the Edman degradation. In this procedure, one amino terminal amino acid residue at a time is removed from a polypeptide to be analyzed. That amino acid is normally identified by reverse phase high performance liquid chromatography (HPLC), but recently mass spectrometric procedures have also been used. The Edman degradation cycle is repeated for each successive terminal amino acid residue until the complete polypeptide has been degraded. The procedure has been automated; and automated sequencers are commercially available through various companies, such as Applied Biosystems, Foster City, Calif. Other techniques, such as the one described in U.S. Pat. No. 6,271,027 to Chait et al. which is incorporated herein by reference, can also be used.

II. DNA Cloning and Sequencing

In accordance with the invention, cDNAs encoding the proteins are also sequenced and cloned. The mRNA can be isolated from the barnacle by homogenizing the tissue in liquid nitrogen; dissolving the frozen tissue in guanidine thiocyanate as described by McCandliss et al., 1981, Methods in Enzymology, 79:51-57, or lysing the frozen tissue in the presence of ribonucleoside-vanadyl complexes and extracting the lysed tissue with phenol as described by Berger et al., 1980, J. Biol. Chem., 255:2955-2961; and purifying poly A-containing RNA by binding on oligo-dT cellulose as described by Aviv, et al., 1972, Proc. Natl. Acad. Sci. U.S.A., 69:1408-1412. Similar procedures may be used in isolating the mRNA transcript of the gene of other marine animals encoding bioadhesive protein.

The mRNA extracted is then employed as a template to produce cDNA in the presence of reverse transcriptase, and dNTPs (dATP, dCTP, dGTP and dTTP), according to well-known procedures, forming a mRNA/DNA hybrid. The mRNA moiety is then removed using known procedures and a second strand of cDNA is synthesized, using the first strand as a template, in the presence of E. coli DNA polymerase I (Klenow fragment), dATP, dCTP, dGTP and dTTP. The double-stranded DNA (ds-cDNA) produced in this manner contains a hairpin loop. The single-stranded loop can conveniently be removed by digestion with S1 nuclease.

Degenerate probes designed based on the N-terminal sequence data from a protein isolated as described above are then used to selectively amplify the gene encoding the protein using the cDNAs as the template. Preferably, this process requires three steps: 1) a 3′ rapid amplification of the cDNA end (RACE); 2) a 5′ RACE; and 3) a clone of the fill length gene. In step 1, degenerate oligonucleotides based on the N-terminal sequence of the selected protein are used in a 3′RACE reaction; and the product of the 3 ′RACE reaction is directly cloned into a vector, e.g. pCR4-TOPO®, Invitrogen, Carlsbad, Calif., and sequenced. In step 2, the sequence of the product of the 3′RACE reaction is used to design internal primers that are used in a 5′RACE reaction; and the 5′RACE reaction product is directly cloned into a vector, e.g. pCR4-TOPO®, Invitrogen, Carlsbad, Calif., and sequenced. Finally, in step 3, primers specific for the beginning and end of the gene, elucidated from the sequences obtained in steps 1 and 2, are used to PCR the full length of the gene using the cDNAs as templates. The full length gene is directly cloned into a vector, e.g. pCR4-TOPO®, Invitrogen, Carlsbad, Calif., and sequenced.

Methods of directly cloning a PCR product into a vector are disclosed in U.S. Pat. No. 5,487,993 and U.S. Pat. No. 5,827,657, both to Herrnstadt et al., which are incorporated herein by reference.

Alternatively, the cDNAs are cloned into cloning vectors in order to produce a clone library to screen for the desired proteins. This screening process requires immobilizing cDNAs on to a nylon membrane and denaturing the cDNAs so that they are single stranded. Labeled degenerate probes are then hybridized to the immobilized cDNA. After washing off the excess and unbound probes, the cDNAs showing positive hybridization are sequenced and analyzed. Current, more sophisticated techniques using microarrays may also be used in the screening method.

DNA sequencing can be accomplished using existing methods. Reviews of DNA sequencing methods, together with future directions and trends, are given by Barrell, 1991, FASEB Journal, 5:40-45; and Trainor, 1990, Analytical Chemistry, 62:418-26. Currently, DNA sequencing is performed by either the chemical degradation method of Maxam et al., 1980, Methods in Enzymology, 65:499-560; or the enzymatic dideoxynucleotide termination method of Sanger et al., 1977, Proc. Natl. Acad. Sci. USA, 74:5463-67.

In the chemical degradation method, base specific modifications result in a base specific cleavage of the radioactive or fluorescently labeled DNA fragment. With the four separate base specific cleavage reactions, four sets of nested fragments are produced which are separated according to length by polyacrylamide gel electrophoresis (PAGE). After autoradiography, the sequence can be read directly since each band (fragment) in the gel originates from a base specific cleavage event. Thus, the fragment lengths in the four “ladders” directly translate into a specific position in the DNA sequence.

In the enzymatic chain termination method, the four base specific sets of DNA fragments are formed by starting with a primer/template system elongating the primer into the unknown DNA sequence area and thereby copying the template and synthesizing a complementary strand by DNA polymerases, such as Klenow fragment of E. coli DNA polymerase I, a DNA polymerase from Thermus aquaticus, Taq DNA polymerase, or a modified T7 DNA polymerase, Sequenase (Tabor et al., 1987, Proc. Natl. Acad. Sci. USA, 84:4767-4771), in the presence of chain-terminating reagents. Here, the chain-terminating event is achieved by incorporating into the four separate reaction mixtures in addition to the four normal deoxynucleoside triphosphates, dATP, dGTP, dTTP and dCTP, only one of the chain-terminating dideoxynucleoside triphosphates, ddATP, ddGTP, ddTTP or ddCTP, respectively, in a limiting small concentration. The four sets of resulting fragments produce, after electrophoresis, four base specific ladders from which the DNA sequence can be determined.

Automated sequencers are also available commercially through various companies, such as Applied Biosystems, Foster City, Calif.

III. Gene Expression

In an embodiment of the invention, the genes for the adhesive proteins are inserted into expression vectors for recombinant production in a host cell. Various recombinant expression systems may be used, such as bacteria, yeast, insect cells/baculovirus, mammalian cells, etc.

The proteins are preferably expressed in a eucaryotic system, such as the insect cells/baculovirus system. In the insect cell/baculovirus system, each gene is cloned into an expression vector using the MaxBac 2.0 kit (Invitrogen, Carlsbad, Calif.). In this kit, the gene is subcloned into a baculovirus transfer vector (pBluBac 4.5 or pBluBac4.5/V5-His) for high-level protein expression. The transfer vector is then cotransfected with Bac-N-Blue linear virus DNA to produce recombinant virus carrying a barnacle adhesion gene. Viral stocks are collected and used for subsequent infections of SF-9 insect cells. The level of protein expression is monitored by Western Blot with a S-Tag HRP LumiBlot Kit (Novagen Inc. Madison, Wis.). The expressed protein is purified using S-Tag rEK Purification Kit (Novagen Inc. Madison, Wis.).

In general, variations in the recombinant gene may be desirable to improve protein function. This is usually accomplished through random mutation and selection. For the present invention, the recombinant gene is preferably about 80% identical to the sequenced gene, more preferably 90% identical to the sequenced gene, and most preferably 95% identical to the sequenced gene.

Likewise, there can be variations in the expressed protein. For the present invention, the expressed protein is preferably about 80% identical to the sequenced protein, more preferably 90% identical to the sequenced protein, and most preferably 95% identical to the sequenced protein.

IV. Antibody Production

In accordance with the invention, antibody specific for each adhesive protein is also produced. The antibodies allow for the detection and analysis of the adhesive proteins.

Conventionally, antibodies have been produced by use of the blood of mammals such as sheep, goats, mice, rabbits, rats, etc. The animal is injected with a protein or fragment of a protein to provide antigenic activity. After a sufficient amount of time, the blood of the animal is harvested and the antibody is collected from the serum by affinity separation.

Limited as it is, the production of antibodies in birds has been utilized. From some points of view, antibody production is more advantageous in birds rather than in mammals. For instance, antibodies (IgY) produced in hens are transferred to eggs and then to chickens hatched therefrom as in mammals whose antibodies (IgG) are transferred from mothers to fetuses. The antibody concentration in the yolk of an egg is higher than or as high as in the blood of the hen. Indeed, the antibodies contained in 300 ml of the blood of a hen which lays 20 eggs in a month are as many as those contained in the eggs. More antibodies can be obtained from the eggs by six to seven times than from 40-50 ml of the blood of a rabbit.

The antibodies may be produced through cell culture methods. Usually, these methods employ a hybridoma to produce the antibody. The process first requires immunizing a mammal, usually a mouse, with an antigen (a protein in this case) to stimulates the production of antibodies against the antigen. The antibody producing cells are then isolated from the animal's spleen and fused with tumor cells growing in cultures forming hybridoma cells. The hibridoma cells can then be grown in culture to produce monoclonal antibody against the antigen. Methods of producing hybridoma cells for antibody production is disclosed in detailed in U.S. Pat. No. 4,196,265 to Koprowski et al., which is incorporated herein by reference.

In a preferred embodiment, antibodies against the barnacle proteins are made by immunizing rabbits with artificially synthesized 18 amino acid peptides. The peptides are identical to the N-terminal sequences of the proteins. Further, the peptides are conjugated to keyhole limpet hemocyanin (KLH) for immunization of rabbits. Before immunization, blood is withdrawn from the rabbit to provide control serum. The rabbits receive four immunizations with blood removal after each immunization. The blood serum is assayed with ELISA with the 18 amino acids peptide linked to BSA as source of antigen. Antibodies are then purified from possitive serum on an affinity column.

The following examples are given to illustrate the present invention. It should be understood that the invention is not to be limited to the specific conditions or details described in these examples.

### EXAMPLE 1

### Isolation of 35 kD Protein

12 μl of hemolymph or base plate sample from the barnacle Semibalanus balanoides were boiled with 5 ml of 4×loading buffer (Novex) and 3 ml of 2-mercaptoethanol and loaded on SDS-PAGE gel (4-15% gradient gel, Novex). The gels were run at 200 volts (constant current) for 30 minutes and stained with Comassie Blue. FIG. 1 is a photograph of the SDS PAGE showing a major protein at 35 kD (lane 1: proteins from the hemolymph of Balanus crenatus; lane 2: proteins from the hemolymph of Semibalanus balanoides; lane 3: proteins from the base plate of Semibalanus balanoides; lane 4: molecular weight markers (Invitrogen, Carlsbad, Calif.)).

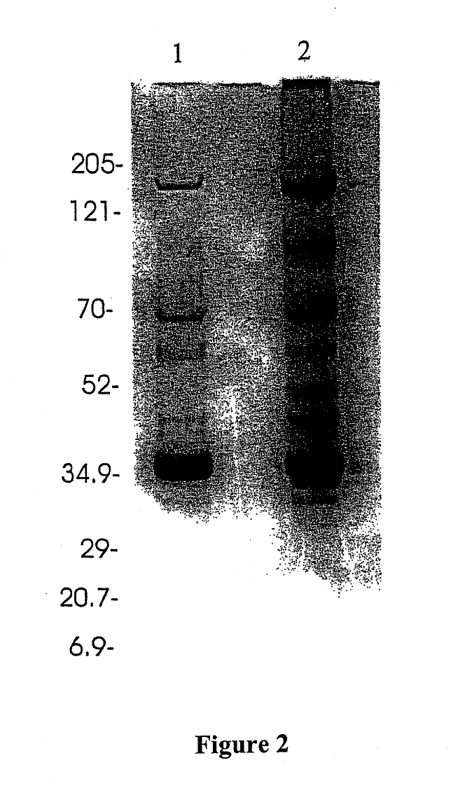

A nickel bead affinity system was then used to isolate the 35 kD protein from the hemolymph and resolved using SDS-PAGE. Several proteins were isolated from the nickel bead system with the most abundant protein having a molecular weight of 35 kD (see FIG. 2; lane 1: proteins purified from the hemolymph using nickel beads; lane 2: total hemolymph proteins).

### Example 2

### Amino Acid Sequence and Structure of the 35 kD Protein

Proteins from the SDS-PAGE were transferred onto PVDF membranes using a blotting system (Novex). The blotting took place at 30 volts (constant current) for 1 hour, after which, the protein bands were visualized with Ponceau stain. The prominent 35 kD band was then cut out of the membrane for sequencing using automated Edman degradation on an Applied Biosystems 477/120 Protein Sequencer. The amino acid sequence for the 35 kD protein is given in SEQ. ID NO. 1.

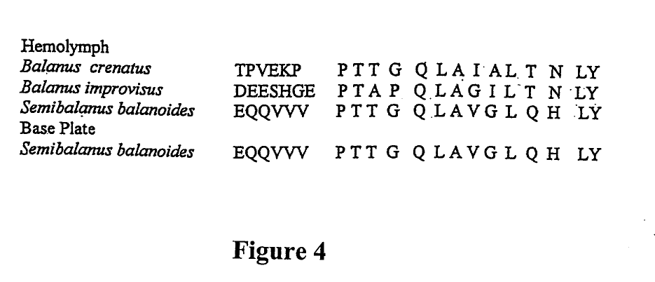

FIG. 4 compares the amino terminal sequence of the 35 kD protein in B. crenatus, B. improvisus, and S. balanoides. The sequences show close homology for all three species.

The 35 kD protein has a calculate molecular weight of 27.9 kD, but migrates electrophoretically at 35 kD on reducing SDS-PAGE. This discrepancy may be due to glycosylation. Indeed, glycosylation staining (GelCode Glycoprotein Staining Kit, Pierce, Rockford, Ill.) of S. balanoides proteins confirmed that the 35 kD protein is glycosylated.

The following glycosylation staining procedure was used. Following SDS-PAGE, gels were fixed in 100 milliliters of 50% methanol for 30 minutes. After fixation, gels were washed in 100 milliliters of 3% acetic acid for 10 minutes with gentle agitation (2×). The wash solution was discarded and 25 milliliters of oxidation solution was added for 15 minutes. The oxidation solution was removed and the gels were washed in 100 milliliters of a 3% solution of acetic acid for 5 minutes (3×). Gels were transferred to 25 milliliters of GelCode glycoprotein staining reagent and gently agitated for 15 minutes. The staining reagent was discarded and 25 milliliters of reduction solution was added for 5 minutes. Finally, gels were washed extensively with 3% acetic acid and with distilled water. Glycoproteins were seen as magenta bands.

### EXAMPLE 3

### Function of the 35 kD Protein

Protein function was determined using sequence homology and motif analysis. A BLAST search of the full amino acid sequence of the 35 kD protein showed homology to filensin, and eye lens protein. Filensin is known to assemble with other proteins to form a filamentous network. The development of a protein filament network is, therefore, likely to be a requirement for base plate formation and application of the glue.

An analysis of the amino acid sequence of the 35 kD protein using the program SignalIP (http://www.cbs.dtu.dk/services/SignalP) confirmed that the first 15 residues of the sequence represents a secretory signal peptide. This finding was consistent with the isolation of the 35 kD protein in the hemolymph and the base plate.

The carboxy terminus region of the 35 kD protein contains a triad repeat of the sequence HDDH. Protein sequences rich in histidine and aspartic acid are known to bind divalent metal ions. This type of interaction may be used by the barnacle to bind ion rich surfaces such as the hull of ships or piping. It may also be used to form protein aggregates by sharing ion binding with other proteins present in the glue or the base plate. Interestingly, the histidine-aspartic acid rich region showed homology to an egg shell protein known to aggregate and cross-link. In addition, this region of the 35 kD protein is also highly homologous to the recent discovered protein pernin, as self-aggregating protein found in the hemolymph of the green lipped mussel.

The 35 kD protein contains a number of methionine residues (8 residues or 3.3% of protein) that may be used to stabilized protein aggregates through methionine oxidation. Such a reaction has been implicated in cataract formation within the eye lens. The use of methionine oxidation is a novel mechanism of stabilizing protein-protein interactions in barnacle base plates or clue. To date, only disulfide cross-linking of cysteine residues and hydrophobic interactions have been suggested as primary mechanisms of stabilizing protein aggregates in barnacle base plate.

### EXAMPLE 4

### Purification of mRNA from Semibalanus balaniodes

Purification of mRNA from S. balanoides was performed by binding poly A-containing RNA to oligo-dT cellulose as described by Aviv, et al., 1972, Proc. Natl. Acad. Sci. U.S.A., 69:1408-1412. Total cellular RNA was precipitated with isopropanol and recovered by centrifugation. The pellet was resuspended in binding buffer (0.5M LiCl, 50 mM sodium citrate, 0.1% SDS) to a final concentration of 1 milligram per milliliter. The solution was incubated at 70° C. for five minutes. The RNA solution was applied to the column (Molecular Research Center, Inc.) and the eluate collected. The eluate was applied again to the column. Messenger RNA (poly A+mRNA) was eluted with 0.6 milliliters of elution buffer (1 mM sodium citrate and 0.1% SDS). One microliter of glycogen and an equal quantity of isopropanol was added to the mRNA. The RNA was precipitated by centrifugation at 12,000×g for 15 minutes. The pellet was washed in 75% ethanol, air dried at room temperature for five minutes and resusupended in 20 microliters. Two microliters of the mRNA solution was used for each cDNA reaction.

### EXAMPLE 5

### RT-PCR

The purified mRNA was used as templates for RT-PCR using the cDNA Cycle® kit (Invitrogen, Carlsbad, Calif.) to form the first strand cDNA. 0.01 to 1 μg was added and mixed with oligo dT primers and heated to 65° C. After centrifugation to collect the reaction mixture, dNTPs and AMV reverse transcriptase was added and incubated at 42° C. for 60 minutes. The mRNA/cNDA hybrids were then denatured by heating at 95° C. for 2 minutes. The reaction mixture was immediately iced to prevent reannealing of the mRNA and cDNA strands.

### EXAMPLE 6

### Amplification and Sequencing of the Gene for 35 kD Protein

This process required four steps: 1) 3′ rapid amplification of the cDNA end (3′RACE); 2) 5′ RACE; 3) clone of the full length gene; and 4) sequencing of the full length gene.

First, the amino terminus sequence of the amino acid sequence given in SEQ. ID NO. 1 was used to design a set of degenerate primers described in SEQ. ID NO. 3. The degenerate primers and the primer for the 3′ end (SEQ. ID. NO. 4) were used in the 3′ RACE reaction using the EDNA produced in example 5 as templates. The product of the 3′RACE reaction was then cloned into a pCR4-TOPO® vector (Invitrogen, Carlsbad, Calif.) and sequenced.

Second, the sequence of the 3′RACE reaction product was then used to design the internal primers of SEQ. ID NOS. 5 and 6 that were used along with an abridged anchor primer in subsequent 5′RACE reactions using the cDNA produced in example 5 as templates. To run the 5′RACE reaction, from about 12 to about 18 cytosines were added to the 5′ end of the cDNA using terminal transferase. The 5′RACE was broken up into two sequential PCR reactions. The first PCR reaction used the primer of SEQ. ID NO. 5 and the abridged anchor primer which comprised a mixture of primers made up of guadinies of about 12 to about 15 bases. The product of the first PCR reaction was then amplified in a second PCR reaction that uses the primer of SEQ. ID NO. 6 and the abridged anchor primer. The reason for the second reaction was to ensure that the desired gene was amplified in the first reaction. For this to take place, the primer of SEQ. ID NO. 5 was located 3′ to the primer of SEQ. ID NO. 6. The product of the 5′RACE reaction was cloned into another pCR4-TOPO® vector and sequenced.

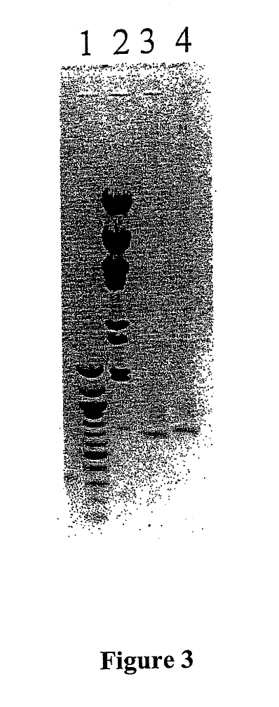

Third, the sequences of both the 3′RACE and 5′RACE reaction product were then used to design primers specific for the beginning (SEQ. ID NO. 7) and the end (SEQ. ID NO. 8) of the gene for the 35 kD protein. These primers were then used to amplify the entire gene using the cDNA produced in example 5 as templates (see FIG. 3; lane 1: 100 DNA Ladder (New England Biolabs, Beverly, Mass.); lane 2: Lambda DNA-BstE II Digest (New England Biolabs, Beverly, Mass.); lane 3: PCR product using primers to the 35 kD protein with no secretory sequence (beginning at position 46 in Seq. ID No. 2); lane 4: PCR product using primers to beginning and end of the gene for the 35 kD protein). Finally, once the gene was selectively amplified from the cDNA, it was cloned into a pCR4-TOPO® vector and sequenced. The gene sequence for the 35 kD protein is given is SEQ. ID NO. 2.

### EXAMPLE 7

### Amplification and Sequencing of the Gene for 4.7 kD Protein

The gene sequencing process required the same steps as those for the 35 kD w protein: 1) 3′ rapid amplification of the cDNA end (3′RACE); 2) 5′ RACE; 3) clone of the full length gene; and 4) sequencing of the full length gene.

First, the amino acid sequence of the amino-terminus of the 4.7 kD protein was determined in a method similar to Examples 1 and 2. The amino-terminus sequence was then used to design a set of degenerate primers of SEQ. ID NO. 11. The degenerate primers and the primer for the 3′ end (SEQ. ID. NO. 12) were used in the 3′ RACE reaction using the cDNA as templates (the cDNA is synthesized according to examples 4 and 5). The product of the 3′RACE reaction was then cloned into a pCR4-TOPO® vector (Invitrogen, Carlsbad, Calif.) and sequenced.

Second, the sequence of the 3′RACE reaction product was then used to design the internal primer of SEQ. ID NO. 14 that was used along with an abridged anchor primer in a subsequent 5′RACE reaction using the cDNA produced in example 5 as templates. Prior to the 5′RACE reaction, about 12 to about 18 cytosines were added to the 5′ end of the cDNA using terminal transferase to provide a site for the abridged anchor primer. The product of the 5′RACE reaction was cloned into another pCR4-TOPO® vector and sequenced.

Third, the DNA sequence for the 4.7 kD protein was determined from the sequences of both the 3′RACE and 5′RACE reaction products and the over lapping regions between the two products. The final DNA sequence for the 4.7 kD protein is given in SEQ. ID NO. 10.

The invention has been disclosed broadly and illustrated in reference to representative embodiments described above. Those skilled in the art will recognize that various modifications can be made to the present invention without departing from the spirit and scope thereof.

What is claimed is:

1. An isolated nucleic acid sequence consisting essentially of Seq. ID No. 2 or Seq. ID No. 10.

2. An isolated nucleic acid sequence comprising Seq. ID No. 2 or Seq. ID No. 10.

3. An isolated nucleic acid sequence which is at least about 80 percent identical to a sequence selected from the group consisting of Seq. ID No. 2 and Seq. ID No. 10, wherein the nucleic acid sequence encodes a barnacle adhesive protein.

4. An isolated nucleic acid sequence encoding the protein of Seq. ID No. 1 or Seq. ID No. 9.

5. The isolated nucleic acid sequence of claim 4, wherein the nucleic acid is selected from the group consisting of DNA and RNA.

6. A vector comprising:
a) the nucleic acid sequence of claim 1; and
b) a promoter operably linked to said nucleic acid sequence, said promoter capable of effecting transcription of said nucleic acid sequence.

7. A vector comprising:
a) the nucleic acid sequence of claim 3; and
b) a promoter operably linked to said nucleic acid sequence, said promoter capable of effecting transcription of said nucleic acid sequence.

8. A vector comprising:
a) the nucleic acid sequence of claim 4, and
b) a promoter operably linked to said nucleic acid sequence, said promoter capable of effecting transcription of said nucleic acid sequence.

9. A microorganism comprising the vector of claims 6, 7, or 8, said microorganism being capable of expressing a barnacle adhesive protein.

10. The microorganism of claim 9, wherein said microorganism is selected from a group consisting of bacteria, yeast, baculovirus, and mamallian cell.

11. The microorganism of claim 10, wherein said bacterial is E. coli.

12. The microorganism of claim 10, wherein said microorganism is baculovirus.

13. An isolated protein consisting essentially of Seq. ID No. 1 or Seq. ID No. 9.

14. The protein of claim 13, wherein the protein cross-links with other proteins.

15. The protein of claim 14, wherein the other proteins are barnacle cement proteins.

16. The protein of claim 15, wherein the barnacle is selected form the group consisting of Semibalanus balanoides and Balanus crenatus.

17. The protein of claim 13, wherein the protein is secreted.

18. The protein of claim 13, wherein the protein is glycosylated.

19. An isolated protein comprising Seq. ID No.1 or Seq. ID No. 9.

20. The protein of claim 19, wherein the protein cross-links with other proteins.

21. The protein of claim 20, wherein the other proteins are barnacle cement proteins.

22. The protein of claim 21, wherein the barnacle is selected form the group consisting of Semibalanus balanoides and Balanus crenatus.

23. The protein of claim 19, wherein the protein is secreted.

24. The protein of claim 19, wherein the protein is glycosylated.

25. An isolated protein consisting of Seq. ID No. 1 or Seq. ID No. 9.

26. The protein of claim 25, wherein the protein cross-links with other proteins.

27. The protein of claim 26, wherein the other proteins are barnacle cement proteins.

28. The protein of claim 27, wherein the barnacle is selected form the group consisting of Semibalanus balanoides and Balanus crenatus.

29. The protein of claim 25, wherein the protein is secreted.

30. The protein of claim 25, wherein the protein is glycosylated.

31. A protein consisting essentially of an amino acid sequence that is at least about 80 percent identical to a sequence selected from the group consisting of Seq. ID No. 1 or Seq. ID No. 9, wherein said protein is capable of cross-linking with other barnacle cement proteins.

32. The protein of claim 31, wherein the protein is secreted.

33. The protein of claim 31, wherein the protein is glycosylated.

34. An antibody capable of specific binding to the protein of claim 31.

35. The antibody of claim 34, wherein the antibody is an antibody fragment.
